(19) United States
(12) Patent Application Publication
(10) Pub. No.: US 2026/0041625 A1
(43) Pub. Date: Feb. 12, 2026

(54) USE OF IXERIS CHINENSIS EXTRACT FOR DELAYING UV-INDUCED SKIN PHOTOAGING, METHOD FOR PREPARING IXERIS CHINENSIS EXTRACT, AND IXERIS CHINENSIS EXTRACT

(71) Applicant: KOI CORP., Taichung City (TW)

(72) Inventors: Cheng-Lin YANG, Changhua City (TW); Wei-Ping HUNG, Lunbei Township (TW); Hung-Chang HUANG, Kaohsiung City (TW); Yi-Chun LEE, Taichung City (TW); Chyong-Woan LIN, Taichung City (TW)

(21) Appl. No.: 18/796,683

(22) Filed: Aug. 7, 2024

(51) Int. Cl. A61K 8/9789; A61Q 19/08

(52) U.S. Cl. CPC A61K 8/9789; A61Q 19/08; A61K 2800/805; A61K 2800/81

(57) ABSTRACT

Disclosed is the use of an Ixeris chinensis extract for delaying UV-induced skin photoaging, a method for preparing Ixeris chinensis extract, and an Ixeris chinensis extract. The preparation method involves preparing Ixeris chinensis coarse material and performing emulsification microwave extraction. The resulting Ixeris chinensis extract has a total flavonoid content of 30000-37000 ppm as measured by an aluminum chloride flavonoid method. This extract can absorb UV light, promote skin fibroblast activity, protect cells from UVA-induced oxidative and DNA damage, and reduce melanin production in cells, thereby preventing or delaying the UV-induced photoaging.

### CROSS-REFERENCE TO RELATED APPLICATIONS

Not applicable.

### BACKGROUND OF THE INVENTION

The present invention relates to an Ixeris chinensis extract. More specifically, it discloses an innovative technology for using an Ixeris chinensis extract to delay ultraviolet-induced skin photoaging, a method for preparing the Ixeris chinensis extract, and the extract itself.

With climate change, the ultraviolet index (UVI) has repeatedly reached new highs. According to the World Health Organization's (WHO) ultraviolet classification standard, a UV index exceeding 6 is considered high, and exposure to sunlight for more than 30 minutes will cause damage. When the UV index exceeds 8, it is considered excessive, and exposure for 15 to 20 minutes will cause skin damage. According to the UV index monitoring results of the Environmental Protection Administration from 2004 to 2009, the proportion of the UV index reaching excessive levels or above exceeded 50% from May to August each year, and according to the statistics of the American Cancer Society, more than 90% of skin cancer cases are associated with sun exposure.

Ultraviolet radiation includes long-wave ultraviolet A (UVA) with wavelengths between 320-400 nm, medium-wave ultraviolet B (UVB) with wavelengths between 280-320 nm, and short-wave ultraviolet C (UVC) with wavelengths between 100-280 nm. The shorter the wavelength of ultraviolet light, the stronger its energy. Therefore, the shortest wavelength of UVC is the most energetic and has considerable destructive power on the DNA of organisms. Fortunately, the ozone layer can completely block UVC from the sun, so organisms on Earth can avoid damage from UVC. Due to the UVC's ability to kill bacteria, viruses, mold, and other microorganisms, it is often used in clinical settings or in water purifiers and dishwashers for disinfection and sterilization.

UVB is the main wavelength band that causes photobiological effects on the skin, leading to erythema, sunburn, redness, skin lesions, skin cancer, etc. Most medium-wave UVB is absorbed by the ozone layer, water vapor, and carbon dioxide as it passes through the atmosphere.

The longest wavelength, UVA, is not absorbed by the atmosphere and passes directly through it. Over 95% of ultraviolet radiation is UVA. This long-wave ultraviolet was previously thought to be harmless to humans. However, recent studies have shown that long-wave UVA is not only the main cause of skin darkening (immediate melanin deposition), but also penetrates deep into the skin, inducing melanin production, causing DNA damage to skin cells, and causing denaturation of molecules such as elastin and collagen, leading to the formation of wrinkles and skin aging phenomena, it is also a major factor in accelerating the progression of skin cancer.

Ultraviolet radiation is the main factor causing skin photoaging. Specifically, large amounts of reactive oxygen species (ROS) are induced in skin tissue exposed to ultraviolet light. These reactive oxygen species will begin to attack various molecules in cells, causing various oxidative damage, including promoting lipid peroxidation on cell membranes, protein oxidation, formation of large amounts of oxidized lipid-protein compounds, and even DNA damage.

With the increasing awareness of health and environmental protection, natural, healthy, and environmentally sustainable raw materials and products are the current development trend in cosmetics and health foods. However, most of the cosmetics, skin care products, and health foods on the market are made with chemically added ingredients. Not only are there numerous types, but many raw materials are derived from petrochemical sources. Long-term use can not only cause skin irritation and affect physical health, but also cause irreversible damage to the ecological environment.

Therefore, it is necessary to develop a natural and safe product that can effectively reduce cell oxidative damage, DNA damage, and melanin production induced by long-wave ultraviolet light.

Ixeris chinensis is a perennial herbaceous plant native to Taiwan, scientifically known as Ixeris chinensis (Thunb.) Nakai. It belongs to the order Asterales, family Asteraceae, genus Ixeris. It is widely distributed in low-altitude mountain areas and fields in Taiwan, also known as “rabbit milk weed” or “goose grass”. It is a commonly used in traditional Chinese herbal medicine and is also one of the raw materials that can be used for food. It is generally used for anti-inflammatory, analgesic and antipyretic effects, abscesses, diarrhea, liver nourishment, and breast cancer. In recent years, many studies have found that it has the ability to inhibit the proliferation of HepG2 liver cancer cells and counteract the inflammation of gastric epithelial cells induced by Helicobacter pylori.

However, there have been no related research reports on the effects of Ixeris chinensis on ultraviolet-induced skin photoaging.

At present, the methods for obtaining an Ixeris chinensis extract are mostly carried out by traditional boiling water extraction or organic solvent extraction. The high-temperature extraction process formed by the boiling water extraction method often causes the structure of the active ingredients to be destroyed and lose activity, and the organic solvent extraction raises safety and environmental concerns. Therefore, it is necessary to improve the extraction method of Ixeris chinensis extract.

### SUMMARY OF THE INVENTION

The main purpose of the present invention is to provide a use of an Ixeris chinensis extract for delaying ultraviolet-induced skin photoaging, a method for preparing the Ixeris chinensis extract, and an Ixeris chinensis extract.

In order to achieve the aforementioned purpose, the present invention adopts the following technical solutions.

A use of an Ixeris chinensis extract for delaying UV-induced skin photoaging, wherein the Ixeris chinensis extract is obtained by extracting an Ixeris chinensis.

A method for preparing an Ixeris chinensis extract, comprising the following steps in sequence.

Preparation of Ixeris chinensis coarse material: a dried Ixeris chinensis raw material is taken, coarsely ground, and sieved into a powdered Ixeris chinensis coarse material.

Emulsification microwave extraction: the Ixeris chinensis coarse material is uniformly mixed with a first solution, then a microwave extraction is performed under the condition of 400 W-500 W output power for 3.5 minutes to 6.5 minutes, followed by a first centrifugation treatment. Take the supernatant and add a second solution, let it stand at 45° C.-50° C. for 25 minutes to 35 minutes for ion equilibration, then perform a second centrifugation treatment. Filter the obtained supernatant and perform a vacuum concentration treatment at 42° C.-65° C. on the filtrate obtained from filtering to obtain the Ixeris chinensis extract.

An Ixeris chinensis extract prepared by the method according to Claim 3, wherein the total flavonoid content measured by an aluminum chloride flavonoid method is 30000 ppm to 37000 ppm.

The Ixeris chinensis extract provided by the present invention can absorb ultraviolet light, promote skin fibroblast activity, protect cells from UVA-induced cell damage including oxidative damage and DNA damage, and can reduce the amount of melanin produced in cells, thereby preventing or delaying ultraviolet-induced photoaging.

### BRIEF DESCRIPTION OF DRAWINGS

FIG. 1 is a flow chart of the method for preparing the Ixeris chinensis extract of the present invention.

FIG. 2 is a comparative diagram of the Ixeris chinensis extract embodiment obtained by performing the preparation method of the present invention and the total flavonoid content of the Ixeris chinensis extract comparative examples obtained by performing the existing preparation method.

FIG. 3A is a component analysis mass spectrogram (I) of the Ixeris chinensis extract embodiment of the present invention.

FIG. 3B is a component analysis mass spectrogram (II) of the Ixeris chinensis extract embodiment of the present invention.

FIG. 3C is a component analysis mass spectrogram (III) of the Ixeris chinensis extract embodiment of the present invention.

FIG. 3D is a component analysis mass spectrogram (IV) of the Ixeris chinensis extract embodiment of the present invention.

FIG. 4A is a molecular structure diagram of the active component (I) of the Ixeris chinensis extract embodiment of the present invention.

FIG. 4B is a molecular structure diagram of the active component (II) of the Ixeris chinensis extract embodiment of the present invention.

FIG. 4C is a molecular structure diagram of the active component (III) of the Ixeris chinensis extract embodiment of the present invention.

FIG. 4D is a molecular structure diagram of the active component (IV) of the Ixeris chinensis extract embodiment of the present invention.

FIG. 5 is a comparative diagram of the cell viability obtained from cytotoxicity tests of the Ixeris chinensis extract embodiment of the present invention and the control group.

FIG. 6 is a UV-visible light spectrum diagram of the Ixeris chinensis extract embodiment of the present invention and the comparative examples.

FIGS. 7A and 7B show the test results of the protective ability of the Ixeris chinensis extract embodiment of the present invention against UVA-induced DNA damage.

FIGS. 8A and 8B show the test results of the protective ability of the Ixeris chinensis extract embodiment of the present invention against UVA-induced oxidative damage.

FIG. 9 shows the ability of the Ixeris chinensis extract embodiment of the present invention to inhibit melanin production induced by melanocyte-stimulating hormone.

FIG. 10 shows the ability of the Ixeris chinensis extract embodiment of the present invention to inhibit UVA-induced melanin production.

### DETAILED DESCRIPTION OF THE INVENTION

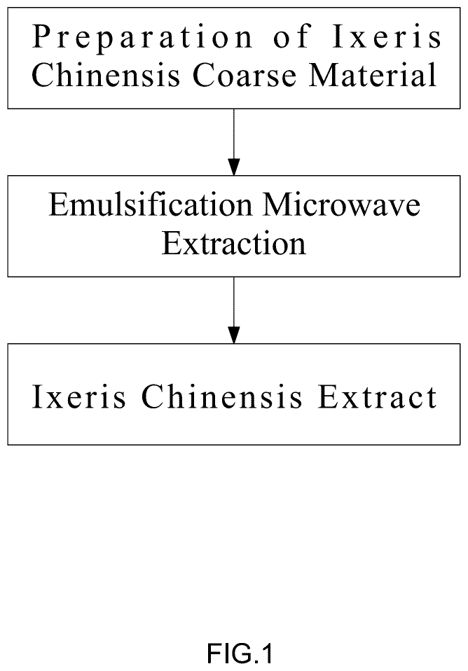

As shown in FIG. 1, the embodiment of a method for preparing Ixeris chinensis extract includes the following steps performed in sequence.

Preparation of Ixeris chinensis coarse material: a dried Ixeris chinensis raw material is taken, coarsely ground, and sieved into a powdered Ixeris chinensis coarse material. In a preferred embodiment, 1 kg of Ixeris chinensis raw material is selected for coarse grinding.

Emulsification microwave extraction: the Ixeris chinensis coarse material is uniformly mixed with a first solution, then microwave extraction is performed under the condition of 400 W-500 W output power for 3.5 minutes to 6.5 minutes, followed by a first centrifugation treatment. Take the supernatant and add a second solution, let it stand at 45° C.-50° C. for 25 minutes to 35 minutes for ion equilibration, then perform a second centrifugation treatment. Filter the obtained supernatant and perform a vacuum concentration treatment at 42° C.-65° C. on the filtrate obtained from filtering to obtain the Ixeris chinensis extract.

Specifically, when the Ixeris chinensis coarse material is mixed with the first solution, the volume ratio of the dried coarse material of the Ixeris chinensis stems and leaves in the Ixeris chinensis coarse material to the first solution is 0.5-1.5:10. The first solution may be water or an aqueous solution containing natural soybean lecithin. When the aqueous solution containing natural soybean lecithin is used as the first solution, the concentration of natural soybean lecithin in the first solution is 7.5-15%.

The preferred microwave output power for microwave extraction is 450 W, and it can optionally perform microwave extraction under the condition of 2400 MHz-2500 MHz frequency. The preferred option for the microwave output frequency is 2450 MHz, and the preferred time for microwave extraction is 4.5 minutes to 5.5 minutes.

The second solution is selected to be a 2.5 mol/1 sodium chloride solution. The preferred option for the standing temperature for ion equilibration is 50° C., and the preferred option for the standing time is 30 minutes.

The acceleration of the first centrifugation treatment is 1400 xg-1600 xg, with the preferred centrifugation acceleration being 1500 xg. The acceleration of the second centrifugation treatment is 3800 xg-4200 xg, with the preferred centrifugation acceleration being 4000 xg.

The supernatant obtained from the second centrifugation treatment is filtered through a membrane with a pore size of 0.45 μm to obtain the aforementioned filtrate.

The flavonoid compounds, also known as flavonoids, are a series of organic compounds characterized by phenolic structures. They are widely distributed in various vegetables, fruits, herbs, grains, nuts, and flowers. Rich flavonoid compounds have also been found in dark chocolate, tea, and red wine. To date, more than five thousand flavonoid compounds have been identified. Flavonoid compounds are divided into six main categories: flavones (such as apigenin and luteolin), flavonols (such as quercetin and myricetin), flavanones (such as naringenin and hesperidin), catechins or flavanols (such as epicatechin and gallocatechin), anthocyanins (such as cyanidin and pelargonidin), and isoflavones (such as genistein and daidzein). Studies have shown that flavonoid compounds have many biological activities, such as antioxidant, anti-inflammatory, anticancer, antimicrobial, neuroprotective, and lipid-lowering benefits.

The Ixeris chinensis extract obtained by carrying out the above method of preparation has a total flavonoid content of 30000 ppm-37000 ppm as measured by an aluminum chloride flavonoid method.

The present invention uses the aluminum chloride flavonoid method to determine the total flavonoid content of the Ixeris chinensis extract. The measurement process is as follows.

In a light-protected environment at room temperature, take 1 ml of the sample to be tested at a concentration of 100 μg/mL, add 9 ml of 95% ethanol, shake uniformly with a test tube shaker to prepare the sample extraction liquid for use.

Take 0.5 mL of the sample extraction liquid, add 100 μl of 5% sodium nitrite, mix well and let it stand for 6 minutes. Then add 100 μl of 10% aluminum chloride, mix thoroughly and let it stand for 6 minutes. Add 1 ml of 5% sodium hydroxide, mix well, and further add 0.8 ml of deionized water and mix well and let it stand at room temperature in the dark for 15 minutes. Then measure the absorbance at a wavelength of 510 nm using a UV-VIS spectrophotometer.

In this measurement, rutin is used as a standard. A calibration curve is plotted using rutin concentrations of 0-0.4 mg/mL to calculate the content of flavonoid compounds in the sample.

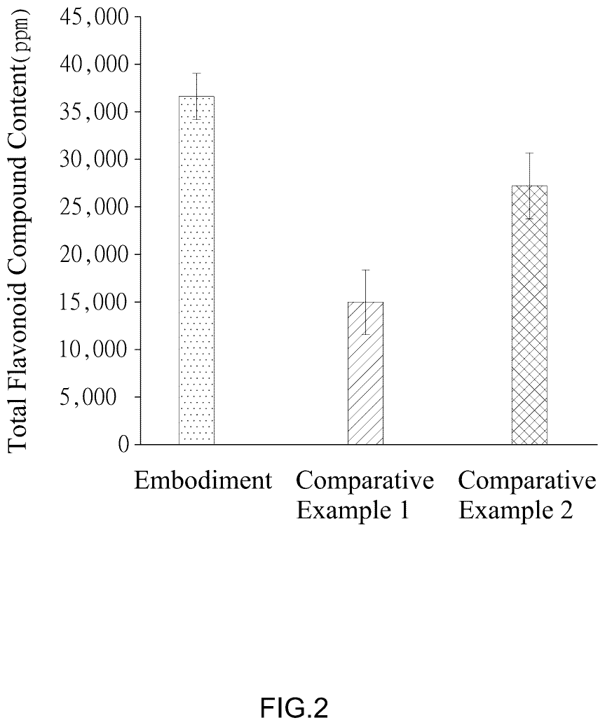

FIG. 2 shows the results of the total flavonoid content measured by the aluminum chloride flavonoid method for a comparative example 1, a comparative example 2, and the Ixeris chinensis extract obtained by carrying out the aforementioned preparation method of the present invention. The comparative example 1 is the Ixeris chinensis extract obtained by a high-temperature and high-pressure extraction method, and the comparative example 2 is the Ixeris chinensis extract obtained by an ultrasonic low-temperature extraction method. The high-temperature and high-pressure extraction method and the ultrasonic low-temperature extraction method are existing technologies familiar to those skilled in the art of the present invention, so their specific steps are not described in detail.

From FIG. 2, it can be seen that the total flavonoid compound content of the comparative example 1 is 17976 ppm, the total flavonoid compound content of the comparative example 2 is 27216 ppm, and the total flavonoid compound content of the Ixeris chinensis extract embodiment of the present invention is 36611 ppm. The method for preparing Ixeris chinensis extract of the present invention is significantly superior to the existing technologies.

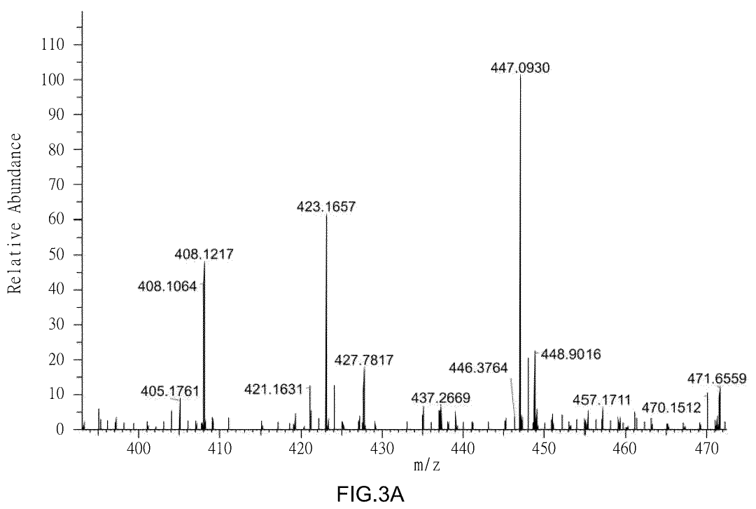

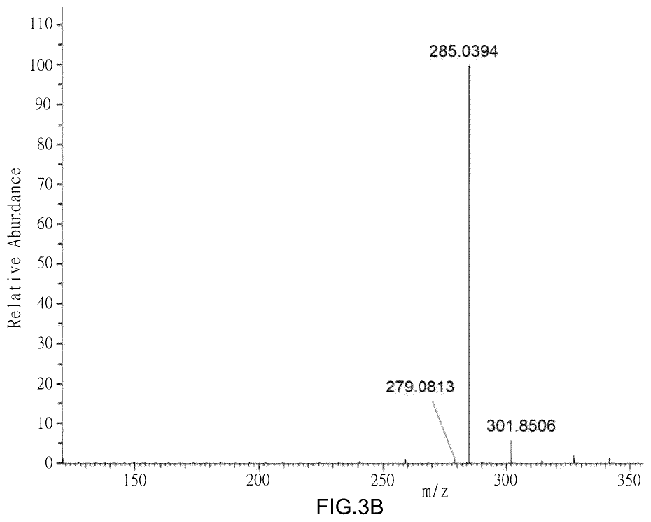

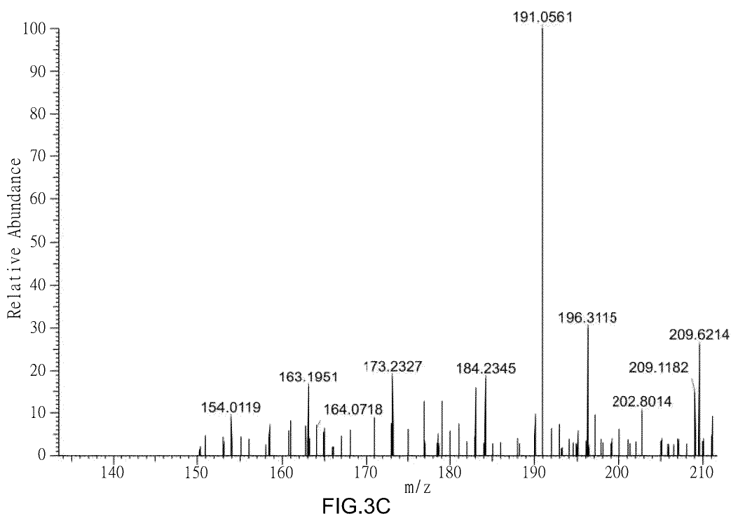

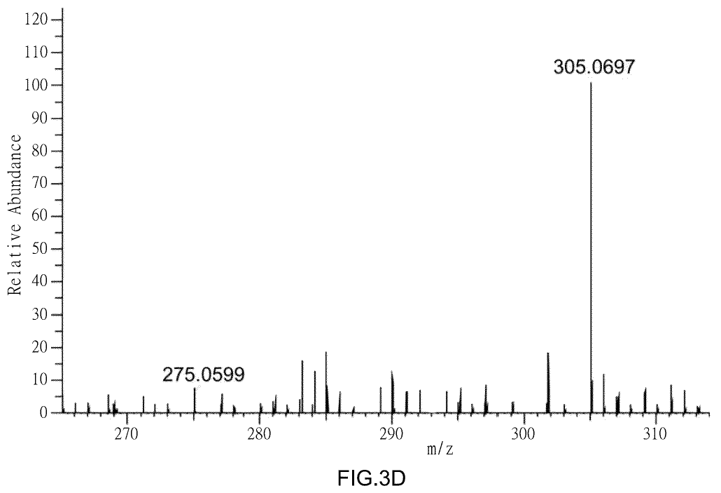

Component analysis of the Ixeris chinensis extract of the present invention was performed using a Fourier-Transform Ion Cyclotron Resonance Spectrometer (FT-ICR MS, also known as FTMS), which is currently the mass spectrometer system with the best mass resolution. It is used in conjunction with an electrospray ionization (ESI) ion source for sample ionization and full mass scanning and continuous mass spectrometry (MSⁿ) experiments. Secondary mass spectrometry is performed according to the mass-to-charge ratio (m/z) of each ion as a basis for ion structure identification. The present invention uses electrospray ionization mass spectrometry (ESI-MS²) in negative ion mode for scanning, with a mass-to-charge ratio (m/z) scanning range of 150 to 2000, to obtain information such as molecular weight and molecular structure of the components in the Ixeris chinensis extract, and to perform structure identification. The main active chemical components and their component analysis results are shown in FIGS. 3A to 3D.

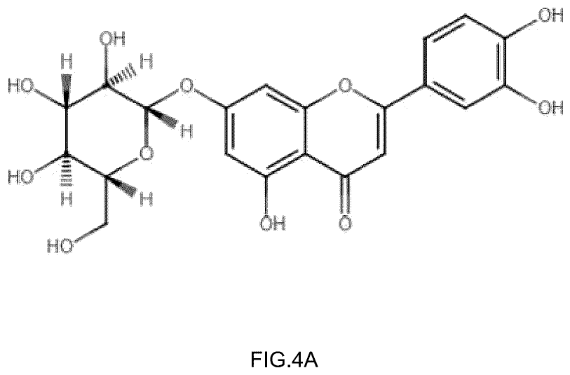

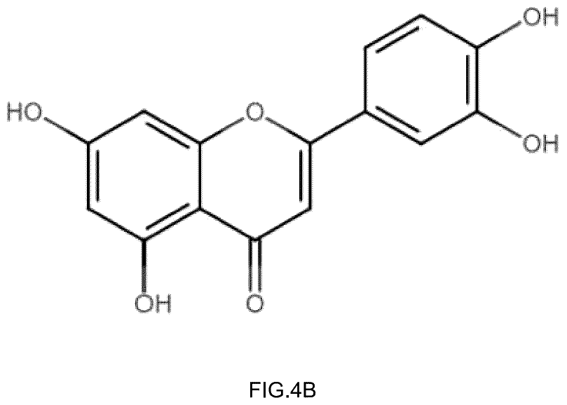

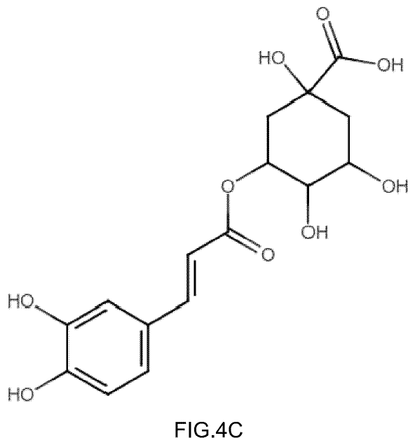

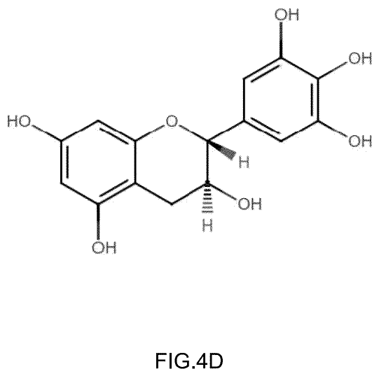

As shown in FIG. 3A, the quasi-molecular ion peak of a compound 1 is m/z 447.0930 [M-H]⁻, and from the mass spectrogram composition, its most likely molecular formula is C₂₁H₁₉O₁₁, with a mass error value of 1.817 ppm. In its ESI-MS² spectrum, the ion m/z 447.0930 loses a hexosyl group and produces ions 285.0394 [M-Glc-H]⁻ and fragment ion m/z 258.7829 [M-H—CHO]⁻. After comparison with the MassBank of North America (MoNA) mass spectrometry database, the compound 1 was identified as luteolin-7-o-beta-D-glucoside, and its molecular structure is shown in FIG. 4A.

As shown in FIG. 3B, the quasi-molecular ion peak of a compound 2 is m/z 285.03936 [M-H]⁻, and from the mass spectrogram composition, its most likely molecular formula is C₁₅H₉O₆, with a mass error value of 2.58 ppm. In its ESI-MS² spectrum, the ion m/z 285.0401 loses a molecule of CO₂ and produces ion m/z 241 and fragment ions m/z 199 [M-H—C₂H₂O—CO₂]⁻, 217 [M-H—C₃O₂]⁻, 175 [M-H—C₂H₂O—C₃O₂]⁻, 243 [M-H—C₂H₂O]⁻. After comparison with the MassBank of North America (MoNA) database, the compound 2 was identified as luteolin, and its molecular structure is shown in FIG. 4B.

As shown in FIG. 3C, the quasi-molecular ion peak of a compound 3 is m/z 353.0860 [M—H]⁻, and from the mass spectrogram composition, its most likely molecular formula is C₁₆H₁₈O₉, with a mass error value of 1.931 ppm. In its ESI-MS² spectrum, fragment ions m/z 191 [QA-H]⁻, 173 [QA-H₂O—H]⁻ are present. After comparison with the MassBank of North America (MoNA) database, the compound 3 was identified as 5-Caffeoylquinic acid, which is a type of chlorogenic acid, and its molecular structure is shown in FIG. 4C.

As shown in FIG. 3D, the quasi-molecular ion peak of a compound 4 is m/z 305.0697 [M—H]⁻, and from the mass spectrogram composition, its most likely molecular formula is C₁₅H₁₄O₇, with a mass error value of 1.83 ppm. In its ESI-MS² spectrum, fragment ion m/z 275.0599 is present. After comparison with the MassBank of North America (MoNA) database, the compound 4 was identified as gallocatechin, which is a monomer component of proanthocyanidin polymers, and its molecular structure is shown in FIG. 4D.

The component analysis results of the aforementioned Ixeris chinensis extract embodiment of the present invention are shown in the following table:

| | Molecular | | Error Value | |
|---|---|---|---|---|
| Number | Formula | [M − H]⁻ | (ppm) | Identification Result |
| 1 | C₂₁H₁₉O₁₁ | 447.0930 | 1.817 | luteolin-7-O-glucoside |
| 2 | C₁₅H₉O₆ | 285.03936 | 2.58 | luteolin |
| 3 | C₁₆H₁₈O₉ | 353.0860 | 1.931 | 5-Caffeoylquinic acid |
| 4 | C₁₅H₁₄O₇ | 305.0697 | 1.83 | gallocatechin |

The cytotoxicity testing is conducted using an in vitro experimental model of mammalian cell toxicity. The evaluation points include cell viability analysis and its effect on appearance changes used to evaluate whether the test sample has potential toxicity to cells.

The cell viability analysis is evaluated using a water-soluble tetrazolium salt (WST-1) cell proliferation assay, which measures cell metabolic activity using a colorimetric method. This method is an indicator of cell viability, proliferation, and cytotoxicity. The effect of the extract on appearance changes is evaluated according to the cytotoxicity evaluation scoring criteria listed in the following table, which is based on ISO 10993-5:2009 (E).

| Grade | Cell Reactivity | Appearance Changes |
|---|---|---|
| 0 | None | No obvious appearance changes compared |
| | | to the blank reagent control group |
| 1 | Slight | <20% of cells show appearance changes or |
| | | become round |
| 2 | Mild | <50% of cells show appearance changes or |
| | | become round |
| 3 | Moderate | <70% of cells show appearance changes or |
| | | become round |
| 4 | Severe | Severe changes, almost all cells show |
| | | morphological changes |

WS1 is a fibroblast cell line isolated from the human body. This WS1 cell line was purchased from the Bioresource Collection and Research Center (BCRC) (Hsinchu, Taiwan) under BCRC number 60300 and was sourced from the American Type Culture Collection (ATCC) under number CRL-1502. The culture medium for the WS1 cells is a minimum essential medium (MEM) containing 1.5 g/I sodium bicarbonate, 0.1 mM non-essential amino acids (NEAA), 1.0 mM sodium pyruvate, and supplemented with 10% (v/v) fetal bovine serum (FBS). Cells are cultured at 37° C. in a cell culture incubator containing 5% carbon dioxide. For subculture, the cells are washed with phosphate-buffered saline (PBS), then an appropriate amount of 0.25% trypsin-EDTA is added and allowed to act for a few minutes at 37° C. The culture flask is gently tapped to detach the cells, then an equal volume of fresh culture medium is added and mixed well. After centrifugation at 1,500 rpm for 5 minutes, the cells are counted and seeded into different culture plates according to the required ratio.

1×10⁴ cells were seeded into 96-well cell culture plates and treated with different concentrations of the Ixeris chinensis extract for an experimental group, different concentrations of dimethyl sulfoxide (DMSO) for a control group, 10% DMSO for a positive control group, or complete culture medium for a blank control group. After culturing at 37° C. for 24 hours, the cells were observed and evaluated using an inverted optical microscope according to the cytotoxicity evaluation scoring criteria listed in the table below. After continued culture for 72 hours, the cell culture medium was removed, the cells were washed with the phosphate-buffered saline, and 2-(4-iodophenyl)-3-(4-nitrophenyl)-5-(2,4-disulfophenyl)-2H-tetrazolium solution (WST-1; Dojindo) was added. After culturing at 37° C. for 2 hours, the absorbance was measured at dual wavelengths of 450 nm and 620 nm using an enzyme-linked immunosorbent assay reader. The cell proliferation ability of the WS1 cells was analyzed by WST-1 detection, and the results were obtained from three independent experiments.

| | Cell | Cell | |
|---|---|---|---|
| Concentration | Viability | Appearance | Cytotoxicity |
| (mg/mL) | (%) | Score | Evaluation |
| 0.05 | 100.38 ± | 0 | No cytotoxicity |
| | 0.17 | | |
| 0.1 | 103.85 ± | 0 | No cytotoxicity |
| | 0.13 | | |
| 0.2 | 108.02 ± | 0 | No cytotoxicity |
| | 3.28 | | |
| 0.4 | 107.17 ± | 0 | No cytotoxicity |
| | 2.22 | | |
| 0.8 | 115.80 ± | 0 | No cytotoxicity |
| | 1.86 | | |
| 1.6 | 120.85 ± | 0 | No cytotoxicity |
| | 4.17 | | |
| Positive | 15.2 ± | 4 | Cytotoxic |
| control | 3.44 | | |
| Blank control | 115.80 ± | 0 | No cytotoxicity |
| | 1.86 | | |

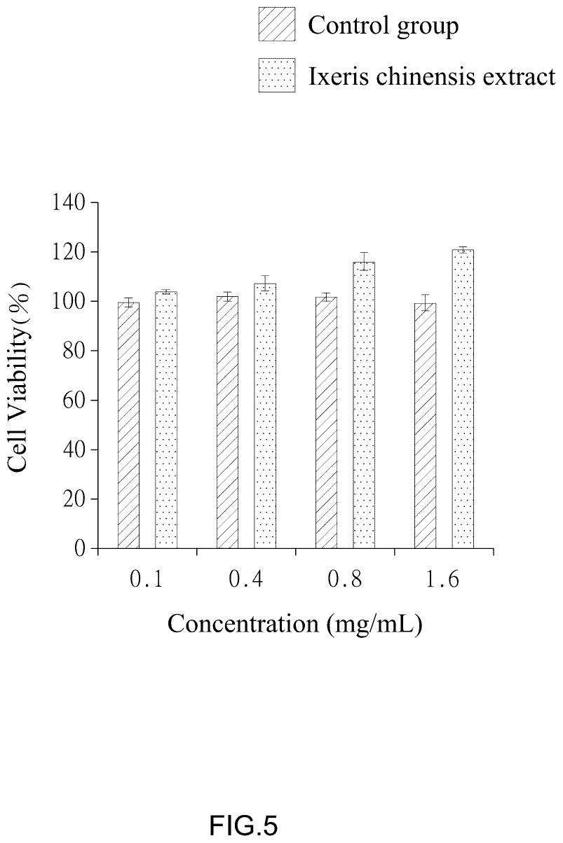

As shown in FIG. 5 and the table above, the results indicate that the Ixeris chinensis extract at concentrations between 0.05 and 1.6 mg/mL does not affect the appearance of the WS1 cells and has no inhibitory effect on their growth, the cytotoxicity evaluation score is grade 0. Therefore, the Ixeris chinensis extract of the present invention is not cytotoxic to normal human fibroblasts.

The ultraviolet light absorption capacity was measured using an ultraviolet-visible spectrophotometer to determine the absorbance of the Ixeris chinensis extract at specific wavelengths to evaluate whether the extract has the ability to absorb ultraviolet light.

The embodiment, the comparative example 1, and the comparative example 2 were each diluted to a 1 mg/mL solution. The diluted Ixeris chinensis extract was placed in a microplate, and absorbance was measured at wavelengths between 200 nm and 600 nm every 10 nm using a SpectraMax 190 full-wavelength plate spectrophotometer from Molecular Devices. The ultraviolet light absorption capacity was evaluated by analyzing the absorbance of the Ixeris chinensis extract at different wavelengths, and the results were obtained from three independent experiments.

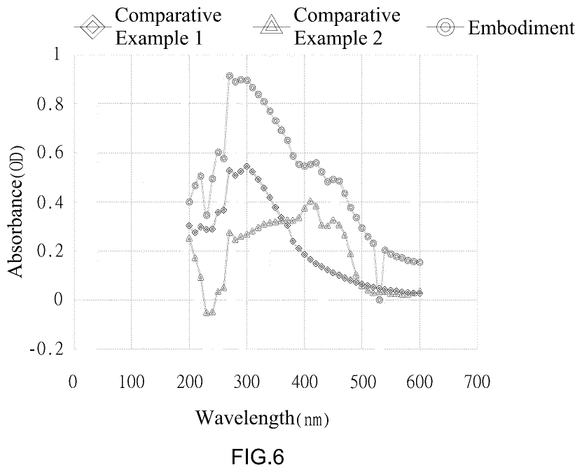

As shown in FIG. 6, the results indicate that the Ixeris chinensis extract embodiment of the present invention has the best absorption capacity between the wavelengths 260 nm and 430 nm, with the lowest absorbance of 0.522 and the highest of 0.914. The comparative example 1 has the best absorption capacity between the wavelengths 260 nm and 350 nm, with the lowest absorbance of 0.362 and the highest of 0.543, and the comparative example 2 has the best absorption capacity between the wavelengths 390 nm and 430 nm, with the lowest absorbance of 0.305 and the highest of 0.402. Therefore, the Ixeris chinensis extract obtained by performing the preparation method of the present invention has a better ultraviolet light absorption capacity compared to the comparative examples 1 and 2 obtained by the existing high-temperature and high-pressure extraction method or the low-temperature microwave extraction method.

The efficacy of the Ixeris chinensis extract in reducing UV-induced DNA damage was evaluated using pUC119 plasmid DNA. The UVA irradiation leads to the generation of free radicals that damage and break circular DNA, resulting in its conversion from a covalently closed circular form to a linear form. This characteristic was used to evaluate the DNA damage protection ability of the Ixeris chinensis extract.

The evaluation method involved mixing 100 ng of pUC119 plasmid DNA with the Ixeris chinensis extract at concentrations of 0 μg/mL, 50 μg/mL, and 100 μg/mL. Each concentration was further divided into two groups. One irradiated with UVA and the other not irradiated. After irradiation, the samples were incubated at 37° C. for 2 hours, followed by gel electrophoresis at 100 V using 1% agarose-TBE gel containing nucleic acid stain (ProTech PT-D1001). The electrophoresis results were photographed under UV light.

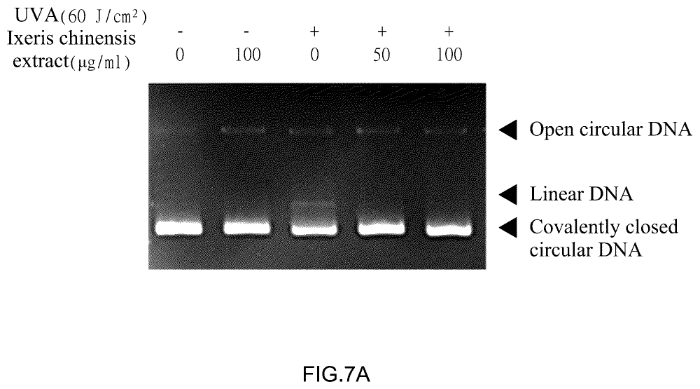

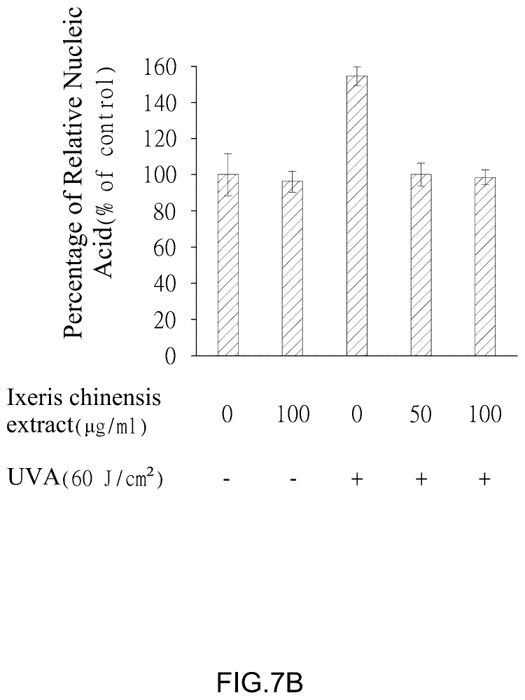

As shown in FIGS. 7A and 7B, the results indicate that the UVA irradiation causes the pUC119 plasmid DNA to transform from a covalently closed circular form to a linear form. However, the pUC119 plasmid DNA treated with 50 μg/mL and 100 μg/mL of the Ixeris chinensis extract did not show linear form formation after UVA irradiation. Thus, the Ixeris chinensis extract of the present invention can effectively protect the pUC119 plasmid DNA from the UVA-induced DNA damage.

The efficacy of the Ixeris chinensis extract in reducing ultraviolet light-induced free radical generation in cells was evaluated using a cutaneous melanoma cell line B16-F10. The amount of free radical generation was measured using a dihydroethidium (DHE) staining assay (Sigma-Aldrich, D7008).

95% of the free radicals in the human body are oxygen free radicals, also known as reactive oxygen species (ROS). The common free radicals include superoxide, hydrogen peroxide, hydroxyl radical, and singlet oxygen. Among these, superoxide is the most abundant free radical in the human body. When the generation and elimination of reactive oxygen species in the body are out of balance, excess reactive oxygen species can cause cellular damage. Dihydroethidium is a fluorescent probe commonly used to detect superoxide free radicals. After entering the cell, it combines with superoxide free radicals to produce 2-hydroxyethidium, which then enters the cell nucleus and intercalates with DNA to emit red fluorescence. This characteristic is used to detect the amount of superoxide free radical generation in cells. The higher the measured fluorescence intensity, the more superoxide free radicals are present in the cells.

B16-F10 cutaneous melanoma cells were seeded in cell culture plates and cultured for 24 hours. Different concentrations (0 μg/mL, 100 μg/mL, and 400 μg/mL) of the Ixeris chinensis extract were added and cultured for 4 hours. The culture medium was removed and the cells were washed with phosphate buffer solution. After irradiation with 9 J/cm² of ultraviolet A (UVA), the medium was replaced with a fresh cell culture medium. Cells not exposed to UVA were used as the control group. After 24 hours of further culture, the cells were washed with phosphate buffer solution, 1 μM dihydroethidium stain was added, and the cells were stained in the dark at 37° C. for 30 minutes. Observations were recorded with the inverted fluorescence microscope, and fluorescence intensity was calculated using Image J software. The results were obtained from three independent experiments.

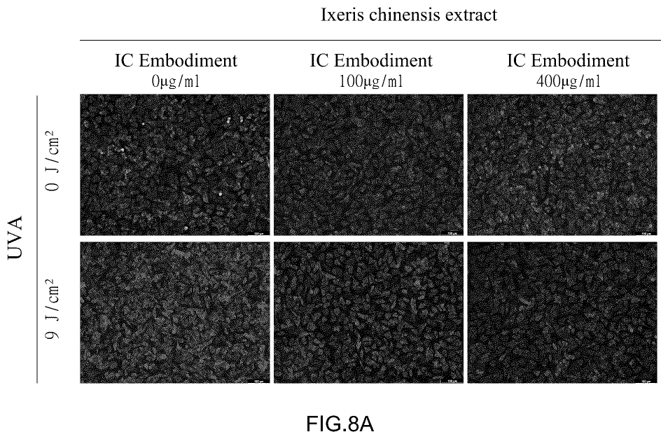

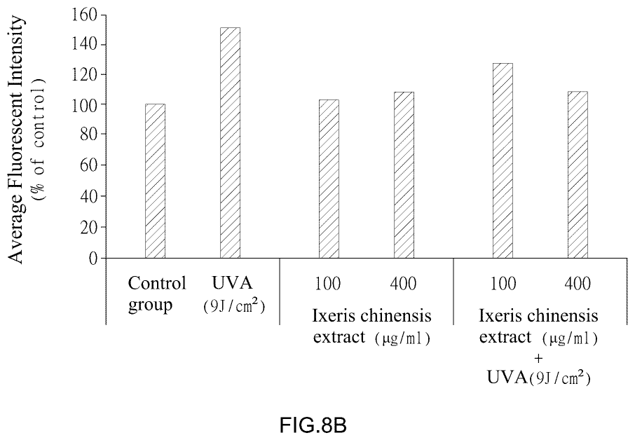

As shown in FIGS. 8A and 8B, the Ixeris chinensis extract at concentrations of 100 μg/mL and 400 μg/mL did not significantly affect the generation of superoxide free radicals in B16-F10 cells. After irradiation with 9 J/cm² of UVA, the superoxide free radicals in the cells increased significantly by 49%, reaching 149%, while in the cells treated with 100 μg/mL and 400 μg/mL of the Ixeris chinensis extract, the UVA-induced superoxide free radical generation was reduced by 23% and 42%, respectively. Based on this, the Ixeris chinensis extract can effectively reduce the generation of superoxide free radicals, thereby achieving protection against UVA-induced cell damage.

The efficacy of the Ixeris chinensis extract in inhibiting melanin production induced by melanocyte-stimulating hormone was evaluated using the cutaneous melanoma cell line B16-F10. B16-F10 cells were seeded in 6-well plates at 2.5×10′ cells per well and cultured for 24 hours. After removing the culture medium and washing the cells with phosphate buffer solution, different concentrations of the Ixeris chinensis extract and 100 nM α-melanocyte-stimulating hormone (α-MSH) were added and cultured for 24 hours. After washing with the phosphate buffer solution, the cells were digested with trypsin and collected by centrifugation, then 1 N NaOH was added. The mixture was reacted at 80° C. for 30 minutes, and the absorbance at 490 nm was measured using the enzyme-linked immunosorbent assay reader. The results were obtained from three independent experiments.

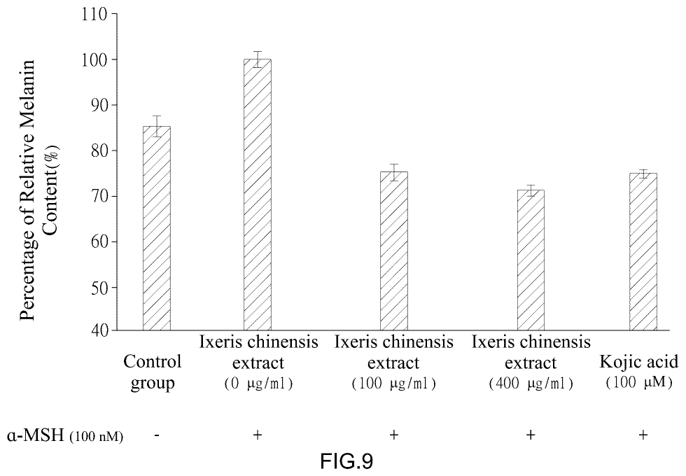

As shown in FIG. 9, the results indicate that the α-MSH treatment induces melanin production in B16-F10 cells. Simultaneous addition of 100 μg/mL or 400 μg/mL of the Ixeris chinensis extract reduced α-MSH-induced melanin production in B16-F10 cells by 23.83% and 27.95%, respectively. Compared with a kojic acid, which was used as a positive control and reduced melanin production by 24.21%, the Ixeris chinensis extract showed better ability to inhibit melanin production. Based on this, the Ixeris chinensis extract of the present invention can effectively reduce melanin production induced by melanocyte-stimulating hormone.

The efficacy of the Ixeris chinensis extract in inhibiting UVA-induced melanin production was evaluated using the cutaneous melanoma cell line B16-F10. B16-F10 cells were seeded in 6-well plates at 2.5×10′ cells per well and cultured for 24 hours. Different concentrations (0 μg/mL, 100 μg/mL, and 400 μg/mL) of the Ixeris chinensis extract or 100 μg/mL kojic acid were added and cultured for 4 hours. The culture medium was removed and the cells were washed with phosphate buffer solution. The cells were then divided into UVA non-irradiated and 9 J/cm² UVA-irradiated groups. After UVA irradiation, fresh cell culture medium was added to each group and cultured for 24 hours. After washing with the phosphate buffer solution, the cells were digested with trypsin and collected by centrifugation, then 1 N NaOH was added. The mixture was reacted at 80° C. for 30 minutes, and the absorbance at 490 nm was measured using the enzyme-linked immunosorbent assay reader, and 1 N NaOH was used as a blank group. The results were obtained from three independent experiments. The percentage of melanin production was calculated as follows: Melanin production percentage (%)=[(absorbance of Ixeris chinensis-treated group—absorbance of blank group)/(absorbance of UVA-irradiated group—absorbance of blank group)]×100%.

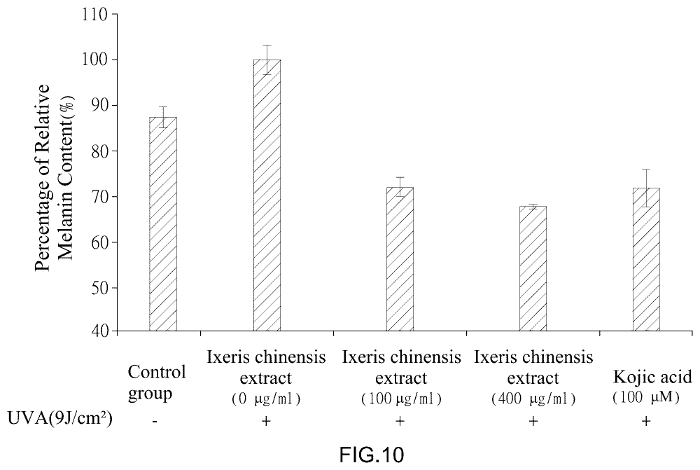

As shown in FIG. 10, the results indicate that the UVA treatment induces melanin production in B16-F10 cells. Addition of 100 μg/mL or 400 μg/mL of the Ixeris chinensis extract reduced UVA-induced melanin production by 28.2% and 32.49%, respectively. Compared with the kojic acid, which was used as a positive control group and reduced melanin production by 28.46%, the Ixeris chinensis extract showed better ability to inhibit melanin production. Based on this, the Ixeris chinensis extract of the present invention can effectively reduce UVA-induced melanin production.

1. A use of an Ixeris chinensis extract for delaying UV-induced skin photoaging, wherein the Ixeris chinensis extract is obtained by extracting an Ixeris chinensis.

2. The use of the Ixeris chinensis extract for delaying UV-induced skin photoaging according to claim 1, wherein the Ixeris chinensis extract is used to reduce UV-induced DNA damage, to reduce free radical generation in cells induced by ultraviolet light, and to inhibit melanin production caused by melanocyte-stimulating hormone, thereby delaying UV-induced skin photoaging.

3. A method for preparing an Ixeris chinensis extract, comprising the following steps in sequence:
preparation of Ixeris chinensis coarse material: a dried Ixeris chinensis raw material is taken, coarsely ground and sieved into a powdered Ixeris chinensis coarse material; and
emulsification microwave extraction: the Ixeris chinensis coarse material is uniformly mixed with a first solution, then a microwave extraction is performed under the condition of 400 W-500 W output power for 3.5 minutes to 6.5 minutes, followed by a first centrifugation treatment, take the supernatant and add a second solution, let it stand at 45° C.-50° C. for 25 minutes to 35 minutes for ion equilibration, then perform a second centrifugation treatment, filter the obtained supernatant and perform a vacuum concentration treatment at 42° C.-65° C. on the filtrate obtained from filtering to obtain the Ixeris chinensis extract.

4. The method for preparing the Ixeris chinensis extract according to claim 3, wherein the first solution is an aqueous solution containing natural soybean lecithin, and the concentration of the natural soybean lecithin is 7.5% to 15%.

5. The method for preparing the Ixeris chinensis extract according to claim 3, wherein the second solution is a sodium chloride solution with a concentration of 2.5 mol/L.

6. The method for preparing the Ixeris chinensis extract according to claim 3, wherein after the Ixeris chinensis coarse material is added to the first solution and mixed uniformly, the microwave extraction is performed under a frequency of 2400 MHz to 2500 MHz.

7. The method for preparing the Ixeris chinensis extract according to claim 3, wherein the acceleration of the first centrifugation treatment is 1400 xg to 1600 xg, and the acceleration of the second centrifugation treatment is 3800 xg to 4200 xg.

8. The method for preparing the Ixeris chinensis extract according to claim 3, wherein the supernatant obtained from the second centrifugation treatment is filtered using a filter membrane with a pore size of 0.45 μm.

9. An Ixeris chinensis extract prepared by the method according to claim 3, wherein the total flavonoid content measured by an aluminum chloride flavonoid method is 30000 ppm to 37000 ppm.

10. The Ixeris chinensis extract according to claim 9, wherein the components of the Ixeris chinensis extract include luteolin glucoside, luteolin, chlorogenic acid, and gallocatechin.
